(19) United States
(12) Patent Application Publication
(10) Pub. No.: US 2020/0085347 A1
(43) Pub. Date: Mar. 19, 2020

(54) MOVEMENT SENSOR

(71) Applicant: 270 Vision Ltd., Hedge End (GB)

(72) Inventor: Martin Gossling, Hedge End (GB)

(21) Appl. No.: 16/494,633

(22) PCT Filed: Mar. 13, 2018
(86) PCT No.: PCT/GB2018/050639

§ 371 Date: Sep. 16, 2019

(30) Foreign Application Priority Data
Mar. 27, 2017 (GB) 1704825.7

(51) Int. Cl. A61B 5/11; A61B 5/00

(52) U.S. Cl. CPC A61B 5/1121; A61B 5/6823; A61B 5/0024; A61B 2562/0223; A61B 5/1126; A61B 2560/0214; A61B 2562/0219; A61B 5/4566

(57) ABSTRACT

A spine movement sensing apparatus is disclosed herein. The spine movement sensing apparatus comprises a string of sensor segments, wherein each sensor segment of the string is configured to attach adjacent to a patient's spine. Each sensor segment comprises at least one sensor for sensing an orientation of the respective sensor segment.

### FIELD OF THE INVENTION

The present disclosure relates to an apparatus and method for sensing movement, for example an apparatus and method for sensing movement of the spine.

### BACKGROUND

Understanding the range of motion of a part of the anatomy such as the spine can be very useful, both for sportspersons in training and recovering from injury, but also the elderly or those persons recovering from surgery including animals such as horses and dogs. Typically all of the low cost available measures of range of motion are subjective and difficult to repeat or verify. However, veterinary surgeons, orthopaedic surgeons, sports scientists, physiotherapists, care homes and general practitioners (GPs) would all greatly benefit from an objective measurement of some kind. Insurance companies and other professional organisations are also looking for ‘Evidence Based Outcomes’ where physical data is now required to prove the effectiveness of any treatment or surgery.

Methods currently being used in the art are very basic, often simply by sight. This makes the data currently available very crude and of poor accuracy and difficult to store and recall. With the increasing use of health insurance to cover physiotherapy and the number of sporting injuries rising, it is clear that better methods need to be found to assess the status of a patient, especially with the requirement for evidence based outcomes.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

### DRAWINGS

Embodiments of the disclosure will now be described, by way of example only, with reference to the accompanying drawings, in which:

FIG. 1 shows a schematic view of an example spine sensing apparatus;

FIG. 2A shows a cross-section of two example segments of a string forming an example spine sensing apparatus;

FIG. 2B shows a perspective view of an example segment for use with a string of segments forming a spine sensing apparatus, such as the spine sensing apparatus of FIG. 1;

FIG. 3 shows a bending (roll) of the mechanical coupling between the two example segments of FIG. 2;

FIG. 4 shows a bending (roll) of a string of segments for use with a spine sensing apparatus;

FIG. 5 shows another example view of a string of segments for use with a spine sensing apparatus;

FIG. 6a shows a twisting (yaw) of the mechanical coupling of the string of segments of FIG. 5;

FIG. 6b shows a bend (pitch) of the mechanical coupling of the string of segments of FIG. 5;

FIG. 7 shows an example spine sensing apparatus comprising a plurality of strings of segments;

FIG. 8 shows a flow chart illustrating a method of fixing a string of sensors to a body for tracking the movement of the body;

FIG. 9 shows a flow chart illustrating a method of fixing a string of sensors to a body for tracking the movement of the body;

FIG. 10 shows a flow chart illustrating a method of determining an orientation of an object for use with determining an orientation of a part of the anatomy of a human or animal body;

FIG. 11 shows a flow chart illustrating a method of determining an orientation of an object for use with determining an orientation of a part of the anatomy of a human or animal body; and

FIG. 12 shows another example spine sensing apparatus.

### SPECIFIC DESCRIPTION

Embodiments of the claims relate to a spine sensing apparatus comprising a string of sensor segments. Each sensor segment of the string is configured to attach adjacent to a patient's spine, and each sensor segment comprises at least one sensor for sensing an orientation of the respective sensor segment. In this way, the degree of mobility in a patient's spine can be objectively assessed, and any areas of limited mobility (for example due to fused discs in the spine) can be accurately determined.

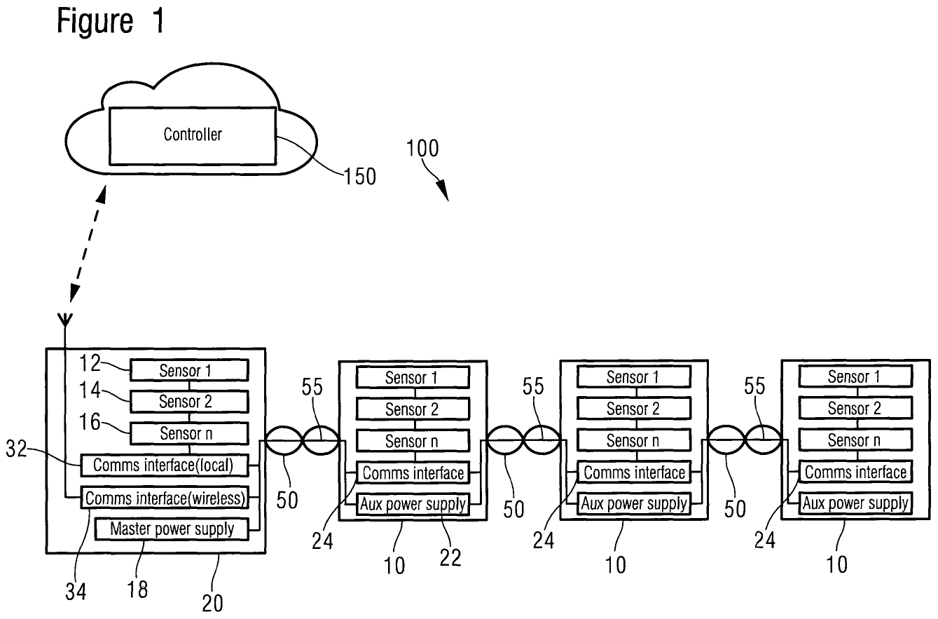

An example spine sensing apparatus is shown in FIG. 1. FIG. 1 shows a string 100 of sensor segments 10. The example string 100 shown in FIG. 1 comprises a master segment 20 and three sensor segments 10, although any number of segments 10, 20 can be used, for example as many as 250 segments 10, 20 may be used. Each segment 10, 20 provides an enclosure for various components that will be discussed in more detail below, and in some examples the enclosure is sealed and washable so that it can be hygienically reused for different patients.

Each sensor segment 10 comprises three sensors comprising a magnetometer 12, an accelerometer 14 and a gyroscope 16 for sensing an orientation of the respective segment 10, 20. Each master segment 20 also comprises three sensors for sensing an orientation of the respective segment 20 comprising a magnetometer 12, an accelerometer 14 and a gyroscope 16. Because the master segment 20 comprises sensors 12, 14 and 16 it may also be considered a sensor segment 10. It will be understood, however, that in other examples each segment 10, 20 may comprise fewer sensors, for example only two sensors such as a magnetometer 12 and an accelerometer 14, or a magnetometer 12 and a gyroscope 16.

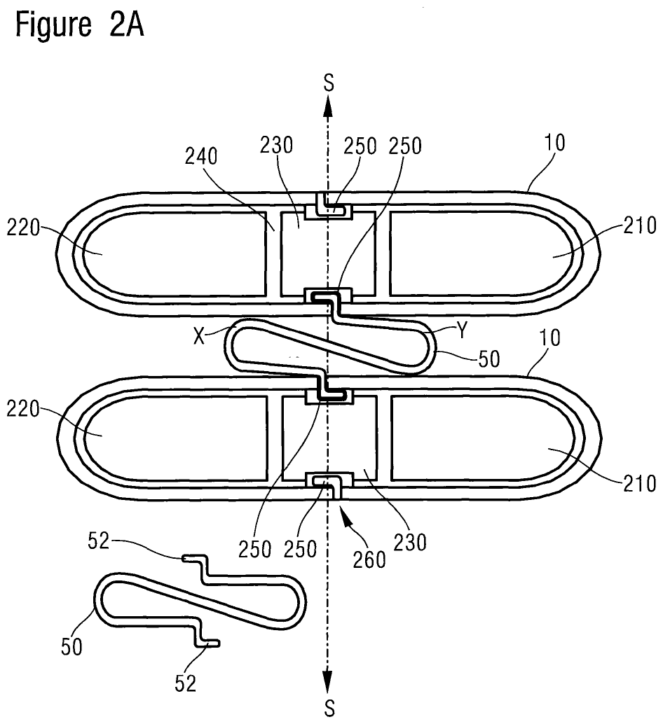

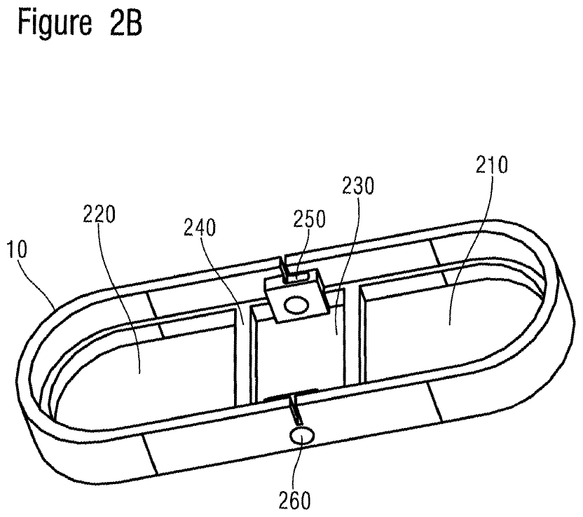

The string 100 of segments 10, 20 are coupled in series via respective mechanical couplings 50. In the example shown in FIG. 1, the mechanical coupling 50 between the segments 10, 20 comprises a non-magnetic, electrically-insulating, resiliently-deformable spring, and in the example shown in FIG. 1 the mechanical coupling is a plastic spring which will be described in more detail with respect to FIGS. 2 to 6b. Providing a non-magnetic, electrically-insulating coupling 50 may be advantageous as it will not interfere with the sensors 12, 14, 16. In particular, a plastic spring will not interfere with a magnetometer.

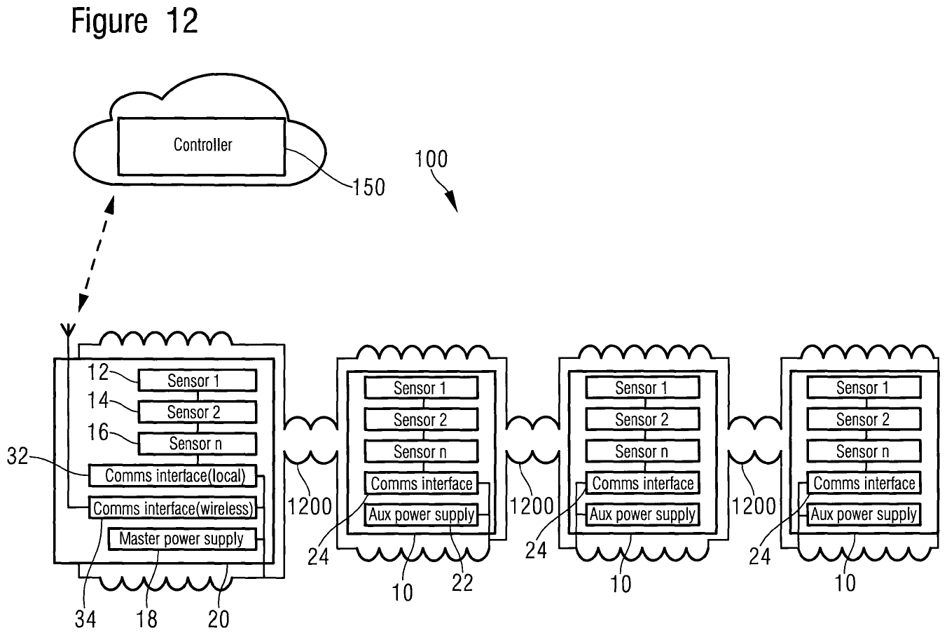

The string 100 of segments 10, 20 are also coupled via an electrical coupling 55 between the segments 10, 20. The electrical coupling comprises at least one physical link connecting each of the sensor segments 10 in series to the master segment 20. It will, however, be understood that in other examples the electric coupling 55 need not be in series but may be arranged in parallel (as shown in FIG. 12), for example. In the example shown in FIG. 1 the electrical coupling 55 is a thin signal wire with a diameter of less than 100 μm, although it will be understood that in other examples the electrical coupling 55 may be formed from a flexible tape or strip, for example or wires of other dimensions. In the example shown in FIG. 1 the electrical coupling 55 travels through the inside of the mechanical coupling 50 so that the mechanical coupling 50 may act to protect or shield the electrical coupling 55.

The master segment 20 comprises a master power source 18 for powering the sensors 12, 14, 16 of the string 100. Each sensor segment 10 also comprises an optional auxiliary power source 22 electrically coupled to the master power source 18 of the master segment 20. The master segment 20 also comprises a string interface 32 and a controller interface 34 coupled to an antenna 36 for communicating wirelessly with a controller 150. The string interface 32 and the controller interface 34 of the master segment 20 are coupled to the master power source 18 and to the first 12, second 14 and third 16 sensors of the master segment 20. Each sensor segment 10 also comprises a string interface 24 coupled to the string interface 24, 32 of an adjacent segment 10, 20. The string interface 24 of each sensor segment 10 is coupled to the first 12, second 14, and third 16 sensors and the optional auxiliary power source 22 of that corresponding segment 10. The string interface 32 of the master segment 20 is coupled to the local communications interface 24 of an adjacent sensor segment 10 of the string 100.

In the example shown in FIG. 1, the string interface 32 of the master segment 20 is configured to communicate with other segments 10 of the string 100. The controller interface 34 comprises a wireless interface for communicating over a wireless network connection and is configured to communicate wirelessly with a controller 150. The string interface 24, 32 of each segment 10, 20 comprises a local network interface for communicating over a physical network connection with adjacent sensor segments 10 and the master segment 20. Each sensor segment 10 is configured to communicate with the master segment 20 via the string interfaces 24, 32.

Communicating via any of the interfaces 24, 32, 34 may comprise sending data comprising information representative of the sensor signals (along with other information such as a unique identifier, as will be described in more detail below), and may be one-way or two-way. For example, the master segment 20 may communicate two-way with a controller 150 and receive signals back from the controller 150 (such as confirmation of receipt), whereas the communication from each of the sensor segments 10 may be one-way.

Each segment 10, 20 is configured to produce sensor signals comprising three dimensional information indicating at least one of the orientation and the location of each respective segment 10, 20. Each respective segment 10, 20 is configured to provide sensor signals defining the orientation of the corresponding segment 10, 20 for determining an orientation of a portion of the spine. The master segment 20 is configured to send these sensor signals from each segment 10, 20 of the string 100 to the controller 150 via the controller interface 34 for determining an orientation of a portion of the spine.

The mechanical coupling 50 between segments 10, 20 is configured to separate the segments 10, 20 at a neutral position and is configured to provide a minimum separation between the segments 10, 20. The mechanical coupling 50 is configured to be resiliently compressible from the neutral position to the minimum separation. The mechanical coupling 50 is also configured to be resiliently extendible beyond the neutral position to increase the separation of the segments 10, 20 beyond the neutral position. In the example shown, the neutral position corresponds to a spacing between the vertebrae of a patient's spine. For example, the neutral position may correspond to an average spacing between vertebrae of an average of the general population. In other examples, the neutral position may correspond to a spacing between vertebrae selected for a particular patient. In some examples, the string 100 may be configured to measure movement of a selected region of the spine, such as a cervical, thoracic or lumbar region, and the neutral position may correspond to an average spacing between vertebrae for that corresponding region.

The mechanical coupling 50 is arranged so that each segment 10, 20 is biased to be parallel to another segment 10, 20 along an axis transverse to the longitudinal axis of the string. The longitudinal axis of the string 100 may correspond to the longitudinal axis of the spine, for example if the string 100 is attached adjacent to a patient's spine. This biasing may help a clinician accurately and repeatably attach the segments 10, 20 adjacent to a patient's spine in the correct orientation. The electrical 55 and mechanical 50 couplings are configured to allow the string 100 to bend and flex with movement of the spine. For example, the electrical 55 and mechanical 50 couplings are configured so as to permit rotational movement of one segment 10, 20 with respect to another segment 10, 20 about a first location and about a second location, wherein the first location and the second location are offset from each other along an axis transverse to the longitudinal axis of the string.

In use, the string 100 is attached to a patient adjacent to their spine (as will be described in more detail below). Once the apparatus is calibrated and running (again, this calibration will be described in more detail below), a patient moves their spine, for example by trying to touch their toes (pitch), or by twisting/leaning left and right (yaw/roll). As the patient moves, the sensors 12, 14, 16 in each segment 10, 20 send sensor signals via their respective string interfaces 24 to the string interface 32 of the master segment 20. The master segment 20 also obtains sensor signals from its own sensors 12, 14, 16.

The sensor signals comprise absolute three dimensional information indicating at least one of the orientation and the location of each segment 10, 20 of the string 100. The sensor signals also comprise a unique identifier identifying the segment 10, 20 (and in some examples the string 100) from which they originate. For example, the sensor signals from each segment 10, 20 comprise a unique MAC address identifying the segment and string from which they originate.

The master segment 20 sends these sensor signals wirelessly (for example via a Bluetooth® connection) via the controller interface 34 to the controller 150. The controller 150 processes these received sensor signals to determine an orientation of a portion of the spine corresponding to the string 100. For example, the controller 150 determines the relative orientation of each segment 10, 20 relative to the other segments 10, 20 using quaternion mathematics, which defines in space the relative position of each segment 10, 20 such that any differential movement in Qx, Qy, Qz and Qw can be determined and then changes measured. Qw defines the 3 dimensional direction the segment 10, 20 is moving in, (imagine dots on the surface of a ball with the segment 10, 20 in the center of the ball, Qw defines which dot on the surface as being the vector of movement the segment 10, 20 is moving towards) with the other parameters defining changes in its axial rotation. The magnetometers 12 are operable to determine the initial degree of twist (displacement in the y axis between adjoining segments 10, 20). The controller 150 may then transform the signals to a three dimensional coordinate space wherein a first dimension in the coordinate space represents a first angle of orientation of the spine (for example pitch), a second dimension in the coordinate space represents a second angle of orientation of the spine (for example yaw) and a third dimension in the coordinate space represents a third angle of orientation of the spine (for example roll) so that the orientation of the spine can be displayed to a clinician/patient via a user interface.

Because the controller 150 receives the unique identifier mapping the sensor signals to each sensor segment 10, 20, the controller 150 knows from where along the string 100 (and optionally from which string 100) each sensor signal originates. This is particularly helpful in the case of a faulty sensor 12, 14, 16 or faulty segment 10, 20 as the controller 150 can identify from which segment 10, 20 the sensor signals are missing and in some cases is operable to interpolate data for the missing sensor signals from that segment 10, 20.

In addition to the sensor signals, in some examples each segment 10, 20 is configured to send a heartbeat signal and/or core body temperature readings and/or other body parameters to the controller 150 and/or to other segments 10, 20 of the string 100. The segments 10, 20 may be configured to send the heartbeat signal if the corresponding segment 10, 20 is operating effectively, so that the controller 150 and/or other segments 10, 20 know if all of the segments 10, 20 of the string 100 are functioning correctly. Additionally or alternatively, the heartbeat signal may comprise information relating to operating conditions of each of the segments 10, 20, for example the operating status of each of the sensors 12, 14, 16 or the auxiliary power source 22.

In some examples, each segment 10, 20 of the string is configured to communicate with at least one other segment 10, 20 of that string 100. For example, each of the sensor segments 10 may be configured to communicate with each other (for example by sending sensor signals and/or a heartbeat signal) in addition to communicating with the master segment 20.

In some examples, at least one sensor segment 10 of the string 100 is configured to send sensor signals from the string 100 to the controller 150 for determining an orientation of a portion of the spine. For example, in some examples, each segment 10, 20 may comprise only a controller interface 34 and not a string interface 32, 24. In such examples, each segment 10, 20 may be arranged to send sensor signals from that respective segment 10, 20 directly to the controller 150, for example by a wireless connection such as a Bluetooth® connection.

In the example shown in FIG. 1, the auxiliary power source 22 of each sensor segment 10 is configured to receive power from the master power source 18 of the master segment 20 via the electrical couplings 55. The master power source 18 is configured to trickle charge each of the auxiliary power supplies 22. In this way, when the apparatus is in use, the sensors 12, 14, 16 are powered by their respective power supplies (so the sensors 12, 14, 16 of each sensor segment 10 are powered by their respective auxiliary power sources 22) but when the apparatus is not in use the auxiliary power supplies 22 are recharged by the master power source 18.

Of course, in some examples, there may be no master power source 18, and each segment 10, 20 has its own respective, independent power source that operates independently of the other power sources. In other examples, there may be no auxiliary power sources 22, and each segment 10, 20 is powered by a single master power source 18 in the master segment 20.

In some examples, the power sources, such as the master power source 18 and/or the auxiliary power sources 22 may be configured to be chargeable by inductive charging, for example each segment 10, 20 may comprise an inductive coil configured to permit inductive charging of a respective power source.

In the examples shown the power sources 18, 22 are rechargeable batteries, such as Ni-MH or Li-Ion batteries with the master power source 18 having a higher power capacity (in terms of mAh) than the auxiliary power sources 22, but it will be understood that some of the power sources, such as the auxiliary power sources 22, may store electrical power capacitively, for example the auxiliary power sources 22 may be capacitors.

Examples of the segments 10, 20 and the mechanical coupling 50 between the segments are shown in more detail in FIGS. 2A to 6b.

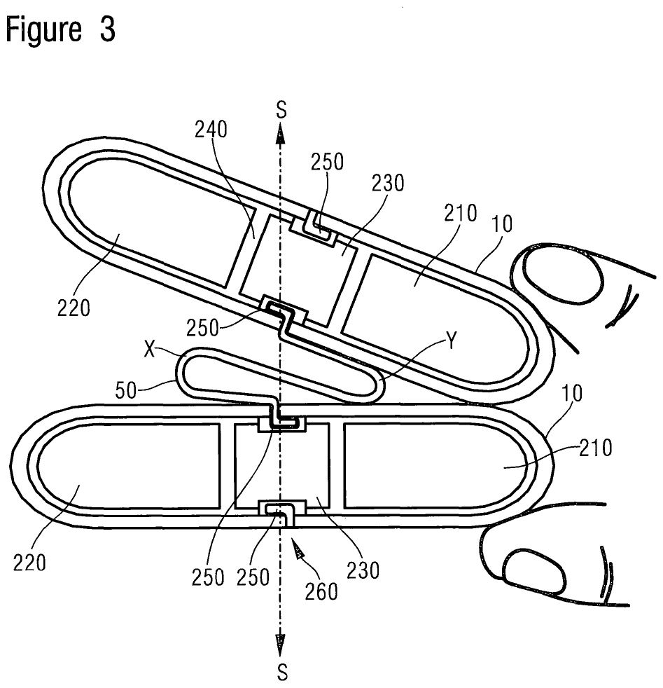

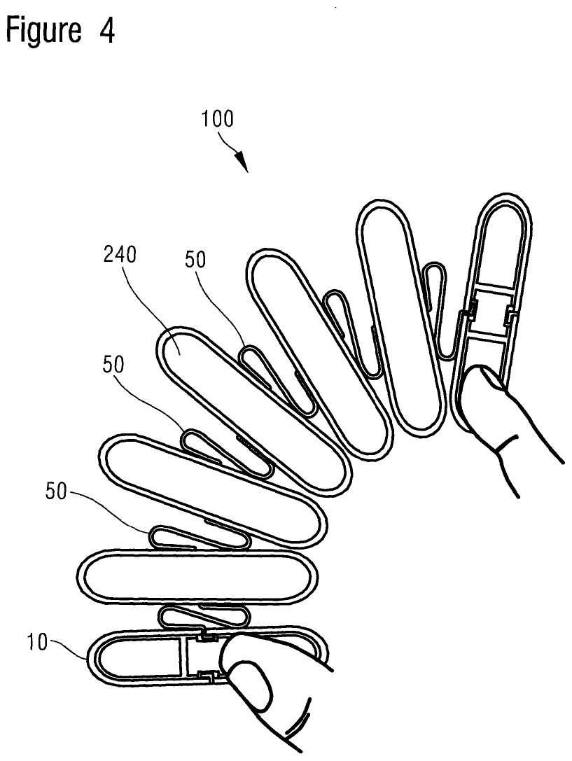

The example segments shown in FIGS. 2A and 2B are sensor segments 10, however it will be understood that equally one of these segments shown in FIGS. 2A and B could be a master segment 20. The body of each of the example segments 10 shown in FIGS. 2 to 6 is substantially oval-shaped, and in the example shown in FIGS. 2A, 2B and 3 has been opened by removing a cover plate to reveal its hollow inside (an example cover plate 270 can be seen in FIG. 4). The segments 10 comprise two lateral regions 210, 220 either side of a central storage region 230. In the example shown, the central storage region 230 is at least 3 mm×3 mm (width and depth) and is arranged to accommodate the sensors 12, 14, 16. Each segment 10 is 50 mm wide and 11 mm deep. The two lateral regions 210, 220 are each arranged to receive a respective battery, and in the example shown each lateral region 210, 220 accommodates a respective 50 mAh battery.

All three regions comprise a shelf 240 extending around the inside perimeter of the segment 10 for supporting a printed circuit board (PCB) comprising the sensors 12, 14, 16, string interface 24 and auxiliary power source 22 mounted thereon. The PCB may be adhered to the shelf 240 so that the components are fixedly attached in the segment 10. The centre of each segment 10 comprises two opposing spring receiving sections 250 on opposite faces of the segment 10 body, each adapted to receive a portion of the mechanical coupling 50. Adjacent to each spring receiving section 250 is an aperture 260 for receiving the electrical coupling 55 therethrough (the electrical coupling 55 is not shown in FIGS. 2 to 6), and in some examples the aperture 260 is arranged to sealingly engage with the electrical coupling 55 so that the segment 10 provides a sealed enclosure. The electrical coupling 55 couples the PCB of one segment 10, 20 with the PCB of an adjacent segment 10, 20 either in series or in parallel. The electrical coupling 55 may comprise four signal wires, each 100 μm in diameter: a ground wire, a positive supply voltage wire, a negative supply voltage wire and a serial bus wire.

Each segment 10, 20 is configured in use to lay horizontally (with respect to a longitudinal axis S of the string, as shown in FIGS. 2 and 3) across the vertebrae of a patient's spine. Each segment 10, 20 may comprise an adhesive pad on each side of the central storage region 230 and adjacent to each lateral region 210, 220 so that the adhesive pads are configured to sit either side of the vertebrae of a patient's spine and attach to the body. Additionally or alternatively the segments 10, 20 may be attached to the patient using medical tape. If the adhesive pads are 5 mm in deep, this which will create a bridge in the middle of the segment 10, 20 which at 5 mm height serves to clear any protruding vertebrae.

In the example shown in FIGS. 2 and 3, the mechanical coupling 50 is in the form of an S-shaped plastic spring that provides a separation of at least 5 mm between segments (although in other examples the mechanical coupling 50 may be configured to provide a separation of at least 2 mm, at least 3 mm, at least 4 mm). The S-shaped spring comprises a hook 52 at each end thereof for insertion into the spring receiving section 250 of a corresponding segment 10, 20. The hook 52 may detachably fasten in the spring receiving section 250 of each segment 10, 20 so that the segments 10, 20 of a string 100 can be interchanged and/or replaced as may be desired.

The mechanical coupling 50 is arranged to be resiliently deformable so that each segment 10, 20 is biased to be parallel to another segment 10, 20 along an axis transverse to the longitudinal axis of the string S, as shown in FIG. 2. The mechanical 50 coupling is configured to allow the string 100 to bend and flex with movement of the spine. As shown in more detail in FIG. 3, the mechanical coupling 50 is configured so as to permit one segment 10 to pivot with respect to another segment 10 about a first location X and about a second location Y, wherein the first location X and the second location Y are offset from each other along an axis transverse to the longitudinal axis of the string S. The example shown in FIG. 3 shows one segment 10 pivoting about a second location Y along an axis transverse to the longitudinal axis of the string S. Such pivoting of the segments 10, 20 with respect to each other allows a string 100 of segments 10, 20 to bend and flex with movement of a patient's spine.

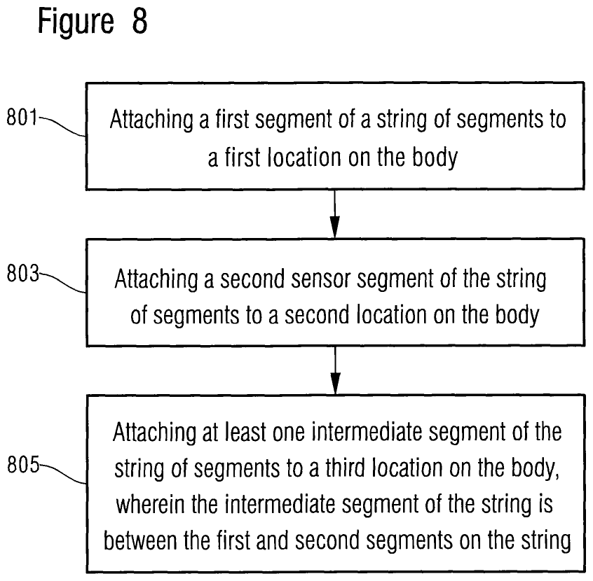

The mechanical coupling 50 between each segment 10, 20 may have the same degree of elasticity, for example the same Young's modulus. For example each mechanical coupling 50 may have the same spring constant (although it will be understood that the mechanical coupling 50 may not necessarily be a spring, but may instead be any material having a degree of elasticity). For example, the material making up each mechanical coupling 50 may have the same bulk modulus and the same shear modulus. By providing a mechanical coupling 50 between each segment 10, 20 of a string of segments 100 having the same Young's modulus, if the first and last segments 10, 20 of a string 100 are fixed to a point, for example adjacent to a patient's spine, and the spine bends, then the mechanical coupling 50 between each segment 10, 20 will bend to the same degree. This will mean that the segments 10, 20 of the string 100 will be evenly spaced out between the first and last segments 10, 20. As will be described in more detail with reference to FIG. 8, having evenly spaced out segments 10, 20 in this way may improve the measurement of the movement of a patient's spine as the distance or separation between each segment 10, 20 may be equal. This may be because it means the segments 10, 20 are evenly spaced along a patient's spine thereby improving the repeatability and accuracy of the measurement of the movement of the spine.

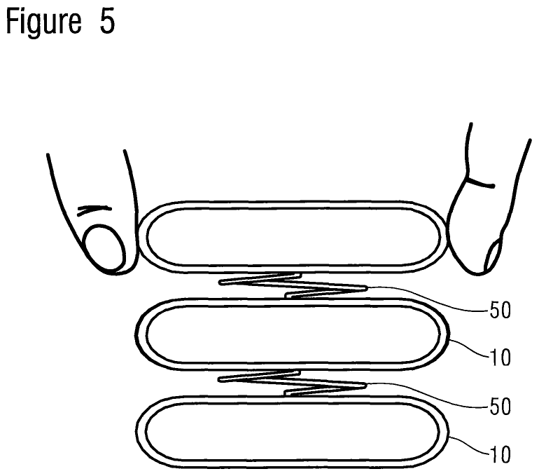

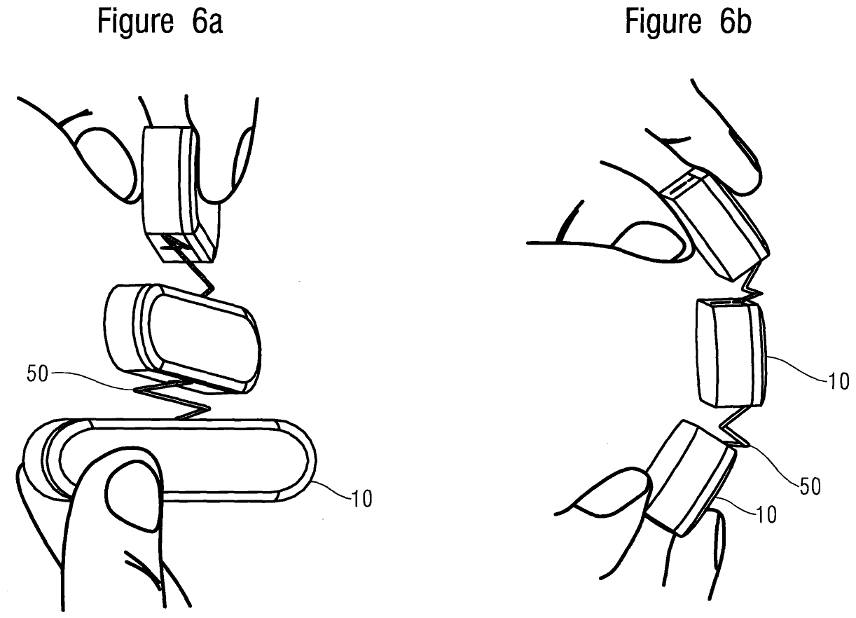

In the examples shown in FIGS. 2 to 4 the mechanical coupling 50 comprises an S-shaped spring, however in other examples the mechanical coupling 50 may comprise an alternative shaped spring or may not comprise a spring at all. For example, the spring may be X-shaped or oval-shaped. In some examples the mechanical coupling 50 may comprise a magazine spring. The springs 50 shown in FIGS. 5, 6a and 6b are Z-shaped, but in other respects are similar to the S-shaped springs described above in relation to FIGS. 2 to 4 as they are configured to allow the string 100 to bend and flex with movement of the spine, as shown in FIGS. 6a and 6b.

The mechanical coupling 50 may be configured to provide a spacing between segments 10, 20 of at least 0.9 mm, at least 1.5 mm, at least 2.1 mm, at least 3.0 mm. Increasing the cross-section of the mechanical coupling 50 increases its stiffness and resistance to twisting. An example cross-section of the mechanical coupling 50 is 1 mm×3 mm.

The segments 10, 20, or any component thereof (such as the springs 50), may be manufactured by subtractive or additive processes. For example, the segments 10, 20 shown in FIGS. 2 to 6 are manufactured using 3D printing using a PLA thermoplastic material. Manufacturing the segments 10, 20 and/or springs 50 in this way may allow a spine movement sensing apparatus to be custom made to a patient's spine to more closely follow the spacing between that particular patient's vertebrae.

The segments 10, 20, or any component thereof, may also be manufactured by assembling pre-manufactured components together such as by adhering a sheetlike element to a substrate. This may be done by laying down a preformed track of the material, or by laying down a larger sheet and then etching it away. This sheetlike element may be grown or deposited as a layer on the substrate. If it is deposited a mask may be used so the deposition happens only on regions which are to carry the track and/or it may be allowed to take place over a larger area and then selectively etched away.

Other methods of manufacture may also be used. For example, the segments 10, 20 and/or springs 50 may be manufactured by way of ‘3D printing’ whereby a three-dimensional model of the segments 10, 20 and/or springs 50 are supplied, in machine readable form, to a ‘3D printer’ adapted to manufacture the segments 10, 20 and/or springs 50. This may be by additive means such as extrusion deposition, Electron Beam Freeform Fabrication (EBF), granular materials binding, lamination, photopolymerization, or stereolithography or a combination thereof. The machine readable model comprises a spatial map of the object to be printed, typically in the form of a Cartesian coordinate system defining the object's surfaces. This spatial map may comprise a computer file which may be provided in any one of a number of file conventions. One example of a file convention is a STL (STereoLithography) file which may be in the form of ASCII (American Standard Code for Information Interchange) or binary and specifies areas by way of triangulated surfaces with defined normals and vertices. An alternative file format is AMF (Additive Manufacturing File) which provides the facility to specify the material and texture of each surface as well as allowing for curved triangulated surfaces. The mapping of the segments 10, 20 and/or springs 50 may then be converted into instructions to be executed by 3D printer according to the printing method being used. This may comprise splitting the model into slices (for example, each slice corresponding to an x-y plane, with successive layers building the z dimension) and encoding each slice into a series of instructions. The instructions sent to the 3D printer may comprise Numerical Control (NC) or Computer NC (CNC) instructions, preferably in the form of G-code (also called RS-274), which comprises a series of instructions regarding how the 3D printer should act. The instructions vary depending on the type of 3D printer being used, but in the example of a moving printhead the instructions include: how the printhead should move, when/where to deposit material, the type of material to be deposited, and the flow rate of the deposited material.

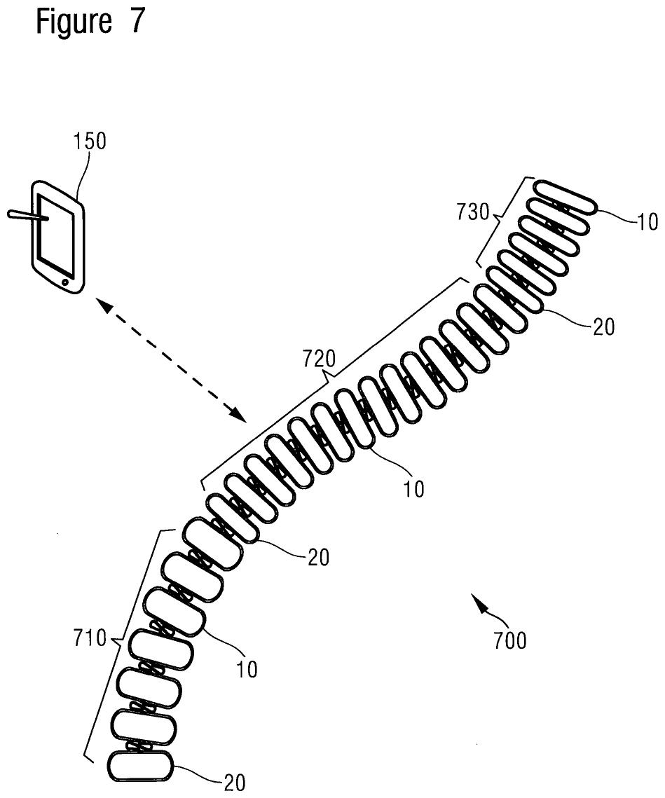

In the examples shown in FIGS. 2 to 6b, each segment 10, 20 is identical in size and dimensions, however in other examples and as shown in FIG. 7, the segments 10, 20 may each be selected to fit around the anthropometric data of a specific patient.

In some examples a range of segments 10, 20 of differing sizes may be provided (for example in the form of a kit) so that a clinician can select the segments 10, 20 (and the total number of segments 10, 20) based on the size (for example height) of a particular patient's spine. In some examples, the mechanical coupling 50 may also be adjusted based on the size of particular patient's spine, for example so that the mechanical coupling 50 matches the spacing between the vertebrae of a patient's spine.

An example spine movement sensing kit 700 comprising a plurality of sensor modules 710, 720, 730 is shown in FIG. 7. Each respective sensor module 710, 720, 730 may comprise a spine movement sensing apparatus 100 as described above. For example, each respective sensor module 710, 720, 730 comprises a respective string 100 of segments 10, 20.

Each respective module 710, 720, 730 is configured to send sensor signals to a controller 150 for determining an orientation of a corresponding respective portion of the spine. Each module 710, 720, 730 is adapted to fit a respective portion of the spine of a human body. For example, as can be seen in FIG. 7, the lower module 710 is adapted to fit a lumbar portion of the spine, the middle module 720 is adapted to fit a thoracic portion of the spine, and the upper module 730 is adapted to fit a cervical portion of the spine.

In the example shown in FIG. 7, the segments 10, 20 of each module 710, 720, 730 are of the same size for each respective module 710, 720, 730, so that each of the segments 10, 20 of the lumbar region are the same size, each of the segments 10, 20 of the thoracic region are the same size, and each of the segments 10, 20 of the cervical region are of the same size. The segments 10, 20 of each module 710, 720, 730 are of different size to each other, so that the segments 10, 20 of the cervical module 730 are smaller than those of the thoracic module 720 which in turn are smaller than those of the lumbar module 710. However, it will be understood that in other examples the segments 10, 20 of a module 710, 720, 730 may vary in size to match the variation in size of the corresponding vertebrae for which they are configured to map, for example so that the segments 10, 20 of the thoracic module get smaller as the distance travelled up the spine towards the cervical region increases.

Similarly, the mechanical couplings 50 between the segments 10, 20 of each module 710, 720, 730 may be the same for each module 710, 720, 730 but differ between modules 710, 720, 730, so that the mechanical coupling 50 is smaller between segments 10, 20 of the cervical module 730 than the thoracic module 720 and the mechanical coupling 50 is smaller between segments 10, 20 of the thoracic module 720 than between segments 10, 20 of the lumbar module.

The lumbar module 710 comprises seven segments 10, 20, the thoracic module 720 comprises fourteen segments 10, 20, and the cervical module 730 comprises six segments 10, 20. In the example shown in FIG. 7 each module is coupled to an adjacent module, for example with a mechanical coupling 50, to form an apparatus extending the length of the spine, however it will be understood that in other examples each module may be separate from (and optionally operate independently of) another module. Also, although each module 710, 720, 730 is shown in FIG. 7 as having a respective master segment 20, in some examples if the modules are coupled together, there may only be one master segment 20 for all of the modules 710, 720, 730. It will also be understood that in other examples the number of segments 10, 20 per module 710, 720, 730 may differ.

The example kit 700 shown in FIG. 7 comprises a controller 150, for example a tablet or laptop computer. The controller 150 is configured to receive sensor signals from each module 710, 720, 730 (for example from a master segment 20 of each module 710, 720, 730). The sensor signals comprise absolute three dimensional information indicating at least one of the orientation and the location of each segment 10, 20 of the each module 710, 720, 730. The sensor signals also comprise a unique identifier identifying the segment 10, 20 the module 710, 720, 730 from which they originate. For example, the sensor signals from each segment 10, 20 comprise a unique MAC address identifying the segment 10, 20 and module 710, 720, 730 from which they originate. The controller 150 is configured to determine an orientation of a portion of the spine corresponding to each module based on the received sensor signals.

As with the apparatus described above in relation to FIGS. 1 to 6b, the master segment 20 of each module 710, 720, 730 in the example shown in FIG. 7 sends these sensor signals wirelessly (for example via a Bluetooth® connection, for example via Bluetooth® meshing) via a controller interface 34 to the controller 150. The controller 150 processes these received sensor signals to determine an orientation of a portion of the spine corresponding to the module 710, 720, 730. For example, the controller 150 determines the relative orientation of each segment 10, 20 relative to the other segments 10, 20, and/or of each module 710, 720, 730 relative to the other modules 710, 720, 730, using quaternion mathematics. The controller 150 may then transform the signals to a three dimensional coordinate space wherein a first dimension in the coordinate space represents a first angle of orientation of the spine (for example pitch), a second dimension in the coordinate space represents a second angle of orientation of the spine (for example yaw) and a third dimension in the coordinate space represents a third angle of orientation of the spine (for example roll) so that the orientation of the spine can be displayed to a clinician/patient via a user interface of the controller 150.

In some examples, each module 710, 720, 730 may not have a segment 10, 20 corresponding to every vertebra. For example, in some examples, a module 710, 720, 730 may have a segment for every other vertebra. In such examples, the controller 150 may be configured to interpolate the orientation of the intermediary vertebrae between segments 10, 20 based on the received sensor signals. For example, a clinician or user may program the controller 150 with the placement of segments 10, 20 on the spine of the user so that the controller 150 knows where on the spine the segments 10, 20 are located.

In some examples a segment 10, 20 may be located on another part of the anatomy. For example, a segment 10, 20 may be attached to a patient's head, shoulder or hips. Such placement of segments 10, 20 on other parts of the anatomy may provide a frame of reference for segments 10, 20 on the spine, for example so that a clinician can determine a range of motion of the spine with respect to the hips or shoulders. In some examples, because the cervical vertebrae are relatively small and have a relatively high range of motion compared to, for example, the thoracic vertebrae, the cervical module 730 may comprise a single segment 10, 20 for attachment to the cervical region of the spine and a single segment 10, 20 for attachment to the head, as it may not be practical to attach a segment 10, 20 to every cervical vertebra.

In some examples, the kit 700 comprises a selection of modules 710, 720, 730 of differing sizes so that a clinician can select the modules most appropriate for the patient. For example, the kit 700 may comprise two cervical modules 730, four thoracic modules 720 and two lumbar modules 710. The kit 700 may also comprise a chart indicating the suitable range over which each module 710, 720, 730 may be used so that a clinician knows which modules to select, for example based on the height of the patient. The kit 700 may be provided in the form of a box or case for easy portability by a user or clinician.

As described above with reference to FIG. 4, in some examples the mechanical coupling 50 between each segment 10, 20 may have the same Young's modulus. Providing a mechanical coupling 50 with the same Young's modulus between segments 10, 20 may allow a string of sensors to be more easily and more accurately attached to a patient's spine. This in turn may improve the repeatability of the measurements.

For example, a method of fixing a string of sensors comprising a plurality of sensor segments mechanically coupled in series and each comprising at least one sensor for sensing an orientation of the respective sensor segment, for tracking the movement of the body, is shown in FIG. 8. The method may comprise attaching 801 a first sensor segment 10, 20 (for example the top segment 10, 20) of the string 100 of segments 10, 20 to a first location on the body. Once the first sensor segment 10, 20 is attached to a first location on the body, a second segment 10, 20 (for example the bottom segment 10, 20) of the string of sensors is attached 803 to a second location on the body. For example, the second segment 10, 20 may be pulled slightly so as to stretch the mechanical coupling 50 between segments 10, 20. The mechanical coupling 50 between each segment 10, 20 may have the same Young's modulus, so that the mechanical coupling 50 between each segment stretches to the same degree thereby spacing out the segments 10, 20 of the string 100 evenly. Once the first and second segments 10, 20 are attached (for example the top and bottom segments 10, 20 of a string 100), at least one intermediate segment 10, 20 of the string 100 of segments 10, 20 can be attached 805 to a third location on the body, wherein the intermediate segment 10, 20 of the string 100 is between the first and second segments 10, 20 on the string 100. Attaching the segments 10, 20 of a string in this way means that the spacing between the segments 10, 20 along the string is even, which may improve the accuracy and repeatability of measurements from the string 100.

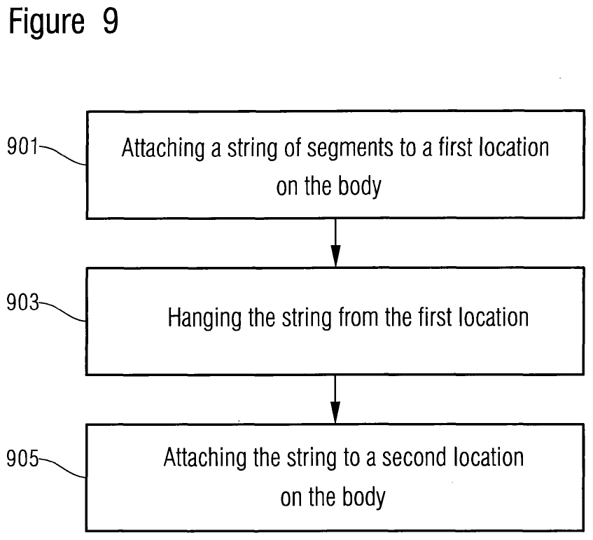

Another example method of fixing a string 100 of sensors comprising a plurality of segments 10, 20 mechanically coupled in series and each comprising at least one sensor for sensing an orientation of the respective segment 10, 20, for tracking the movement of the body, is shown in FIG. 9. The method may comprise attaching 901 a string 100 of segments 10, 20 to a first location on the body. Once the string 100 is attached to the first location on the body, the string 100 of segments 10, 20 are hung 903 via the mechanical coupling 50 from the first segment 10, 20, for example, so that the mechanical coupling 50 between the segments 10, 20 of the string 100 stretches slightly. The mechanical coupling 50 between each segment 10, 20 may have the same Young's modulus, so that the mechanical coupling 50 between each segment stretches to the same degree thereby spacing out the segments 10, 20 of the string 100 evenly. The method may then comprise attaching 905 the string 100 to a second location on the body.

For example, the method may comprise attaching 901 a first segment 10, 20 of the string 100 of segments 10, 20 to a first location on the body. Once the first segment 10, 20 is attached to the first location on the body, the string 100 of segments 10, 20 are allowed to hang 903 via the mechanical coupling 50 from the first segment 10, 20, for example, so that the mechanical coupling 50 between the segments 10, 20 of the string 100 stretches slightly. The method may then comprise attaching 905 another segment 10, 20 of the string 100 hanging via the mechanical coupling 50 to a second location on the body.

Before the string 100 or kit 700 is used to determine the movement of a spine, it may need to be initially calibrated. The calibration may be performed by a controller 150, such as the controller 150 described above in relation to FIGS. 1 and 7. The calibration may comprise using a first sensor providing absolute orientation information to initially determine an orientation of the segments 10, 20 of a string 100 as a reference point. Once the initial reference is obtained using the first sensor, relative movement of the segments 10, 20 relative to the reference point may be determined using a second sensor or a combination of the first and second sensors (or more sensors) to determine a change in orientation of the segments 10, 20. For example, the first sensor may comprise a magnetometer and the second sensor may comprise an accelerometer and/or a gyroscope. Using a combination of sensor signals in this way may provide a more accurate determination of movement of the spine.

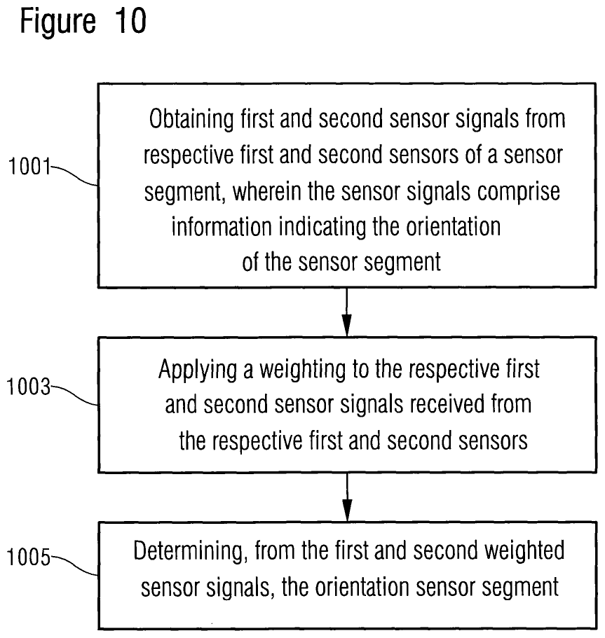

FIG. 10 shows a method of determining an orientation of an object for use with determining an orientation of a part of the anatomy of a human or animal body. The method may be performed by a controller 150, such as the controller 150 described above in relation to FIGS. 1 and 7. The method shown in FIG. 10 comprises obtaining 1001 first and second sensor signals from respective first and second sensors of a segment (such as a segment 10, 20 described above in relation to FIGS. 1 to 7), wherein the sensor signals comprise information indicating the orientation of the segment 10, 20.

Once the first and second sensor signals are obtained, a weighting is applied 1003 to the respective first and second sensor signals received from the respective first and second sensors, and the orientation of the segment 10, 20 is determined 1005 from the first and second weighted sensor signals,.

The first sensor signals may comprise sensor signals comprising information defining an absolute orientation of the segment 10, 20 with respect to a fixed position, for example with respect to a magnetic pole. For example, the first sensor may comprise a magnetometer. The second sensor signals comprise information defining a change in orientation of the sensor segment with respect to time. For example, the second sensor may comprise an accelerometer or a gyroscope.

The method may further comprise obtaining the first and second sensor signals over a time interval, adjusting the weighting as function of the time interval, applying the adjusted weighted to the received sensor signals, and determining a change in position and/or orientation of the segment 10, 20 over the time interval based on the weighted sensor signals. A first weighting may be applied for a first time interval and a second weighting may be applied for a second time interval. For example, the first sensor signals may be favoured during the first few seconds of use so that for the first time interval the first sensor signal from the first sensor is dominant in the determination of the orientation, and afterwards the second sensor signals may be favoured so that for the second time interval the second sensor signal from the second sensor is dominant in the determination of the orientation of the segments 10, 20.

In other examples, the weighting may be adjusted as a function of relative movement. For example, if the sensor segment 10, 20 is determined to be relatively stationary (for example by at least one of the sensors 12, 14, 16), the first sensor signals may be favoured, but if movement is detected then the second sensor signals may be favoured.

As described above in relation to FIGS. 1 to 7, the segments 10, 20 may comprise a third sensor. In such examples, the method may further comprise obtaining third sensor signals from the third sensor of the sensor segment, applying a weighting to the third sensor signal, and determining, from the first, second and third weighted sensor signals, the orientation of the sensor segment.

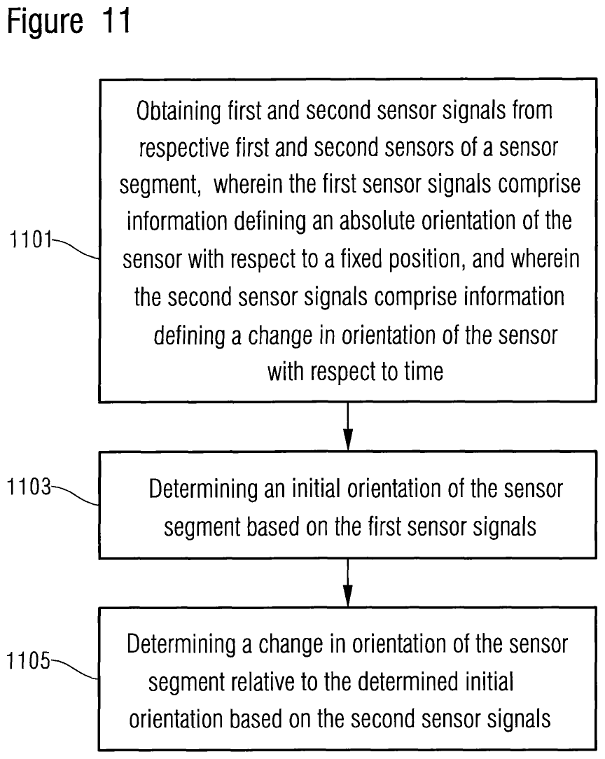

An example method of determining an orientation of an object for use with determining an orientation of a part of the anatomy of a human or animal body is shown in FIG. 11. The method shown in FIG. 11 comprises obtaining 1101 first and second sensor signals from respective first and second sensors of a segment 10, 20, wherein the first sensor signals comprise information defining an absolute orientation of the sensor with respect to a fixed position, and wherein the second sensor signals comprise information defining a change in orientation of the sensor with respect to time. Once the first and second sensor signals are obtained, an initial orientation of the segment 10, 20 based on the first sensor signals is determined 1103, and a change in orientation of the segment 10, 20 relative to the determined initial orientation is determined 1105 based on the second sensor signals.

As described above with reference to FIG. 10, determining a change in orientation of the segment 10, 20 relative to the determined initial orientation may comprise determining a change in orientation of the segment 10, 20 based on a combination of the first and second sensor signals relative to the determined initial orientation.

The spine movement sensing apparatus described above in relation to FIGS. 1 to 7 may be configured to perform a method of determining an orientation of an object as described above. For example, the spine movement sensing apparatus of any of FIGS. 1 to 7 may comprise an accelerometer and a magnetometer as sensors, and a controller (such as the controller 150 described above) configured to determine an orientation of the sensor apparatus when stationary based primarily on the magnetometer and to determine orientation of the sensor during movement based primarily on the accelerometer. Additionally or alternatively, the spine movement sensing apparatus may comprise a magnetometer and a gyroscope as sensors, and a controller configured to determine an orientation of the sensor apparatus when stationary based primarily on the magnetometer and to determine orientation of the sensor during movement based primarily on the gyroscope.

Also disclosed herein is a sensor apparatus comprising a magnetometer, an accelerometer and a controller (such as the controller 150 described above). The controller is configured to determine an orientation of the sensor apparatus when stationary based primarily on the magnetometer and to determine orientation of the sensor during movement based primarily on the accelerometer. The sensor apparatus may further comprise a gyroscope and the controller is configured to determine orientation of the sensor during movement based primarily on the accelerometer and the gyroscope.

In some examples the controller is configured to receive sensor signals from the magnetometer and the accelerometer, and is configured to determine an orientation of the sensor apparatus when stationary based primarily on the magnetometer by applying a weighting to the sensor signals that favours the sensor signals received from the magnetometer. The controller is then configured to determine, from the weighted sensor signals, the orientation of the sensor apparatus.

Also disclosed herein is a sensor apparatus comprising a magnetometer, a gyroscope and a controller (such as the controller 150 described above). The controller is configured to determine an orientation of the sensor apparatus when stationary based primarily on the magnetometer and to determine orientation of the sensor during movement based primarily on the gyroscope. The sensor apparatus may further comprise an accelerometer and the controller is configured to determine orientation of the sensor during movement based primarily on the accelerometer and the gyroscope.

In some examples the controller is configured to receive sensor signals from the magnetometer and the gyroscope, and is configured to determine an orientation of the sensor apparatus when stationary based primarily on the magnetometer by applying a weighting to the sensor signals that favours the sensor signals received from the magnetometer. The controller is then configured to determine, from the weighted sensor signals, the orientation of the sensor apparatus.

In some examples, in response to the sensor apparatus returning to stationary after movement, the controller is configured to determine the orientation of the sensor apparatus based primarily on the magnetometer.

In some examples, the controller is configured to determine the orientation of the sensor apparatus based increasingly on the magnetometer as the speed of movement of the sensor apparatus decreases.

In some examples, the controller is configured to determine the orientation of the sensor apparatus based increasingly on the accelerometer as the speed of movement of the sensor apparatus increases. Additionally or alternatively, the controller is configured to determine the orientation of the sensor apparatus based increasingly on the gyroscope as the speed of movement of the sensor apparatus increases.

FIG. 12 shows another example spine sensing apparatus. The apparatus shown in FIG. 12 is in many respects similar to the spine sensing apparatus of FIG. 1 (with the same or similar reference numbers indicating features with the same or similar functionality), but instead of respective mechanical 50 and electrical 55 couplings between segments 10, 20, the string 100 comprises a combined coupling 1200 between segments 10, 20. The combined coupling 1200 may be resiliently deformable, as with the mechanical coupling 50 described above in relation to FIGS. 1 to 6b, and may extend around the outside of each intermediate segment 10 of the string 10. In-between each segment 10, 20, the combined coupling 1200 may be arranged to have a number of folds or bends, so as to permit flexion, bending and stretching of the coupling 1200 between segments 10, 20 of the string 100.

The combined coupling 1200 may be configured to pass electronic signals, such as sensor signals over the combined coupling 1200 in addition to providing a source of power for the sensors 12, 14, 16 of each segment 10, 20. For example, the combined coupling 1200 may comprise two couplings, one coupling one side of the string and another coupling another side of the string, with the two couplings providing respective positive and negative power sources to which the segments are coupled in parallel. The sensor signals may be sent over such a coupling 1200 via known methods, such as via powerline communication (PLC). The string interface 24, 32 of each segment 10, 20 may therefore comprise a DC/AC filter configured to send the sensor signals over the combined coupling 1200.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

Other variations and modifications of the apparatus will be apparent to persons of skill in the art in the context of the present disclosure. Although the above examples have been described in terms of measuring the movement of the spine, it will be understood that they could equally be applied to other parts of the anatomy or even to other objects (such as buildings, sporting equipment, vehicles and so on).

1. A spine movement sensing apparatus comprising:
a string of sensor segments, wherein each sensor segment of the string is configured to attach adjacent to a patient's spine; and wherein each sensor segment comprises at least one sensor for sensing an orientation of the respective sensor segment.

2. The apparatus of claim 1 wherein the string comprises a master segment comprising (i) a string interface for communicating with other segments of the string, and (ii) a controller interface for communicating with a controller.

3. The apparatus of claim 2 wherein the string interface comprises a local network interface for communicating over a physical network connection, and the controller interface comprises a wireless interface for communicating over a wireless network connection.

4. The apparatus of claim 3 wherein the master segment comprises a power source for powering the at least one sensor of each segment of the string.

5. The apparatus of claim 1 wherein the string comprises a master segment comprising a power source for powering the at least one sensor of each segment of the string.

6.-36. (canceled)

37. A method of determining an orientation of an object for use with determining an orientation of a part of the anatomy of a human or animal body, the method comprising:
obtaining first and second sensor signals from respective first and second sensors of a segment, wherein the sensor signals comprise information indicating the orientation of the sensor segment; applying a weighting to the respective first and second sensor signals received from the respective first and second sensors; and
determining, from the first and second weighted sensor signals, the orientation of the sensor segment.

38. The method of claim 37 wherein the first sensor signals comprise sensor signals comprising information defining an absolute orientation of the sensor segment with respect to a fixed position, and wherein the second sensor signals comprise information defining a change in orientation of the sensor segment with respect to time.

39. The method of claim 38 further comprising:
obtaining the first and second sensor signals over a time interval;
adjusting the weighting as function of the time interval;
applying the adjusted weighted to the received sensor signals; and determining a change in position and/or orientation of the sensor segment over the time interval based on the weighted sensor signals.

40. The method of claim 39 wherein a first weighting is applied for a first time interval and a second weighting is applied for a second time interval.

41. The method of claim 40 wherein the weighting is selected so that for the first time interval the first sensor signal from the first sensor is dominant in the determination of the orientation and for the second time interval the second sensor signal from the second sensor is dominant in the determination of the orientation.

42. The method of claim 41 wherein the sensor segment comprises a third sensor, the method further comprising obtaining third sensor signals from the third sensor of the sensor segment, applying a weighting to the third sensor signal, and determining, from the first, second and third weighted sensor signals, the orientation of the sensor segment.

43.-47. (canceled)

48. A sensor apparatus comprising: a magnetometer, a gyroscope and a controller; wherein the controller is configured to determine an orientation of the sensor apparatus when stationary based primarily on the magnetometer and to determine orientation of the sensor during movement based primarily on the gyroscope.

49. The sensor apparatus of claim 48 wherein the sensor apparatus further comprises an accelerometer and the controller is configured to determine orientation of the sensor during movement based primarily on the accelerometer and the gyroscope.

50. The sensor apparatus of claim 49 wherein: the controller is configured to receive sensor signals from the magnetometer and the gyroscope; and
wherein the controller is configured to determine an orientation of the sensor apparatus when stationary based primarily on the magnetometer by applying a weighting to the sensor signals that favours the sensor signals received from the magnetometer; and
wherein the controller is configured to determine, from the weighted sensor signals, the orientation of the sensor apparatus.

51. The sensor apparatus of claim 50 wherein in response to the sensor apparatus returning to stationary after movement, the controller is configured to determine the orientation of the sensor apparatus based primarily on the magnetometer.

52. The sensor apparatus of claim 51 wherein the controller is configured to determine the orientation of the sensor apparatus based increasingly on the magnetometer as the speed of movement of the sensor apparatus decreases.

53. The sensor apparatus of claim 45 wherein the controller is configured to determine the orientation of the sensor apparatus based increasingly on the accelerometer as the speed of movement of the sensor apparatus increases.

54. The sensor apparatus of claim 48 wherein the controller is configured to determine the orientation of the sensor apparatus based increasingly on the gyroscope as the speed of movement of the sensor apparatus increases.

55. (canceled)

56. (canceled)
